(19) United States
(12) Patent Application Publication
(10) Pub. No.: US 2014/0222160 A1
(43) Pub. Date: Aug. 7, 2014

(54) METHOD OF USING MEDICAL IMPLANTS

(71) Applicant: The Regents of the University of California, Oakland, CA (US)

(72) Inventor: Takahiro OGAWA, Torrance, CA (US)

(73) Assignee: The Regents of the University of California, Oakland, CA (US)

(21) Appl. No.: 14/150,665

(22) Filed: Jan. 8, 2014

Related U.S. Application Data
(63) Continuation of application No. PCT/US2012/045625, filed on Jul. 5, 2012.
(60) Provisional application No. 61/505,891, filed on Jul. 8, 2011.

(51) Int. Cl. A61L 27/36; A61F 2/30

(52) U.S. Cl. CPC A61L 27/3683; A61F 2/30767
USPC 623/23.57

(57) ABSTRACT

Disclosed herein is a method of using a medical implant in a subject. The method comprises treating a medical implant with ultraviolet light (UV) in a closed environment, causing the temperature of the medical implant to be between room temperature (Rt) and about 37° C., and immediately, and placing the implant of a temperature from about the room temperature to about 37° C. in a site in need within the subject.

### RELATED APPLICATION

This application is a continuation of International Application No. PCT/US2012/045625, filed on Jul. 5, 2012 and entitled “METHOD OF USING MEDICAL IMPLANTS,” which in turn claims priority to U.S. Provisional Application 61/505,891 filed on Jul. 8, 2011 and entitled “REACTIVATION OF HIGH ENERGY AND CELL-ATTRACTIVE IMPLANT MATERIALS,” each of which is hereby incorporated by reference herein in its entirety.

### BACKGROUND OF THE INVENTION

1. Field of the Invention

This invention generally relates to a medical implant for biomedical use. In particular, the present invention relates to methods of activating medical implant materials.

2. Description of the Background

Reconstruction and repair following femoral neck fracture, degenerative changes of knee and hip joints and missing teeth are quite common procedure and have considerable medical and societal impact. We experience 300,000 incidence of hip fracture alone in the US, and annual expenditures for treating the osteoporotic fractures are estimated at $13.8 billion [1]. Titanium is a proven biocompatible material, and the use of titanium implants as an endosscous anchor has become essential in such treatments.

Despite the growing needs of titanium implants, a decent percentage of unsuccessful implants, for instance, ranging 5%-40% in orthopedic implants [2-5], and limited application due to unfavorable host site anatomy [6-10], and protracted healing time of implants, particularly in dental implants, are the immediate challenges. Furthermore, the implant placement, facing often times the impaired bone regenerative potential, such as osteoporotic and aged metabolic properties, increase the level of difficulty to achieve the biological requirements of bone-titanium integration [7, 9-11]. Therefore, technologies to enhance the bioactivity of titanium surfaces are desired.

Successful implant anchorage is dependent upon the magnitude of bone directly contacting the titanium surface without soft/connective tissue intervention, which is referred to bone-titanium integration or osseointegration. To ensure the successful bone-implant integration, it is essential that bone-making cells, such as osteoblasts, osteoprogenitor cells, or stem cells, need to attach and adhere to implant surfaces. Recent studies demonstrated that new titanium surface or titanium surfaces immediately after processing are significantly bioactive, as represented by the increased attachment and function of bone-making cells (osteoblasts), leading to the remarkably enhanced bone formation around the surface [12, 13]. These new surfaces are known to be very hydrophilic, on which the contact angle of water is near 0°, which is referred to as superhydrophilic. However, the new titanium surfaces lose the hydrophilicity over time and accordingly decrease its bioactivity and bone making capability [12, 13]. Titanium surfaces stored for 4 weeks since processing become hydrophobic and show only less than 50% capability to attract osteoblasts compared to newly processed surfaces.

Another recently made pivotal discovery in the field of implants is that UV treatment of titanium surfaces recovers the degraded biological capability of aged titanium surfaces [14, 15]. UV treatment makes old hydrophobic surfaces superhydrophilic and increases the level of cell attraction and other osteoconductive capability to the equivalent to or higher than the level of the new surfaces. Therefore, the following would be a plausible strategy and unprecedented benefit for the users and patients to obtain more promising clinical outcomes; titanium implants should be delivered to the peripheral users within certain tolerable days after recovering them by UV treatment at the manufactures. The UV-enhanced titanium surfaces may possess a reasonable level of bioactivity which is around 70% of the new surfaces within 1 week [12].

Regardless of the use in dental and orthopedic therapy, implant products are sold in the storable device in a sterilized package with either air or liquid (such as water or saline solution). During the inventory, transportation, and circulation, the implant products are advertently and unavoidably in the low- or high-temperature conditions (lower or higher than room temperature, i.e., approximately 25° C.). The implant products are also often exposed in low or/and high temperature during the storage at the peripheral user levels, such as in the dental office and orthopedic hospital. Thus, the drastic temperature change is a nearly unavoidable event to happen for implant products in the current medical and commercial system. It is virtually impossible for implant products to be delivered and used for patients without being exposed in the temperature lower or higher than the regular room temperature.

The embodiments described below address the above identified issues and needs.

### SUMMARY OF THE INVENTION

In one aspect of the present invention, it is provided a method of placing an implant in a subject, which method comprising:

treating a medical implant with ultraviolet light (UV) in a closed environment,

causing the temperature of the medical implant to be between room temperature (Rt) and about 37° C., and immediately thereafter

placing the implant of a temperature from about the room temperature to about 37° C. in a site in need thereof in the subject.

In some embodiments of the method, the medical implant has a temperature or is exposed to a temperature below room temperature (Rt) or above body temperature prior to receiving the UV treatment.

In some embodiments of the method, the medical implant has a temperature or is exposed to a temperature between 0° C. and about 20° C. prior to receiving the UV treatment.

In some embodiments of the method, the medical implant has a temperature or is exposed to a temperature of 40° C. or above prior to receiving the UV treatment.

In some embodiments of the method, causing the temperature of the medical implant to be between room temperature (Rt) and about 37° C. comprises the act of heating (e.g., heating by the UV treatment) or cooling.

In some embodiments of the method of invention, optionally in combination with any or all of the various above embodiments, the closed environment is a closed chamber.

In some embodiments of the method of invention, optionally in combination with any or all of the various above embodiments, the closed environment is a closed chamber filled with an inert gas, clean air, or carbon-free air.

In some embodiments of the method of invention, optionally in combination with any or all of the various above embodiments, the inert gas comprises N2, He, or Ar.

In some embodiments of the method of invention, optionally in combination with any or all of the various above embodiments, the medical implant comprises a metallic material.

In some embodiments of the method of invention, optionally in combination with any or all of the various above embodiments, medical implant comprises a surface comprising a micro or nanostructures.

In some embodiments of the method of invention, optionally in combination with any or all of the various above embodiments, the metallic material comprises gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, or combinations thereof.

In some embodiments of the method of invention, optionally in combination with any or all of the various above embodiments, the medical implant is selected from the group consisting of tooth medical implants, jaw bone medical implant, repairing and stabilizing screws, pins, frames (e.g., mesh frames), and plates for bone, spinal medical implants, femoral medical implants, neck medical implants, knee medical implants, wrist medical implants, joint medical implants such as an artificial hip joint, maxillofacial medical implants such as ear and nose medical implants, limb prostheses for conditions resulting from injury and disease, and combinations thereof.

In some embodiments of the method of invention, optionally in combination with any or all of the various above embodiments, the medical implant comprises a non-metallic material.

In some embodiments of the method of invention, optionally in combination with any or all of the various above embodiments, the non-metallic material comprises a polymeric material or a bone cement material.

In some embodiments of the method of invention, optionally in combination with any or all of the various above embodiments, the bone cement material comprises a material selected from the group consisting of polyacrylates, polyesters, bioglass, ceramics, calcium-based materials, calcium phosphate-based materials, and combinations thereof.

In some embodiments of the method of invention, optionally in combination with any or all of the various above embodiments, the bone cement material comprises poly(methyl methacrylate) (PMMA) or methyl methacrylate (MMA).

In some embodiments of the method of invention, optionally in combination with any or all of the various above embodiments, the subject is a mammal.

In some embodiments of the method of invention, optionally in combination with any or all of the various above embodiments, the subject is a human being.

In some embodiments of the method of invention, optionally in combination with any or all of the various above embodiments, the subject has a bone related condition, wherein the method treats or ameliorates the disorder.

In some embodiments of the method of invention, optionally in combination with any or all of the various above embodiments, the bone related condition is a bone related disease or injury.

### BRIEF DESCRIPTION OF THE DRAWINGS

FIG. 1 shows test results by photos on titanium disks of storage in air at different storage temperatures.

FIG. 2 shows the summary of test results on titanium disks of storage in air at different storage temperatures.

FIG. 3 shows test results by photos on titanium disks of storage in liquid at different storage temperatures.

FIG. 4 shows the summary of test results on titanium disks of storage in liquid at different storage temperatures.

FIG. 5 shows test results by photos on a fresh titanium disk and this disk after storage in air after different length of time.

FIG. 6 shows the summary of test results on a fresh titanium disk and this disk after storage in air after different length of time.

FIG. 7 shows test results by photos on a fresh titanium disk and this disk after storage in liquid after different length of time.

FIG. 8 shows the summary of test results on a fresh titanium disk and this disk after storage in liquid after different length of time.

FIG. 9 shows test results on capability of cell attraction on old titanium disks stored in air with and without UV treatment.

FIG. 10 shows test results on capability of cell attraction on old titanium disks stored in liquid with and without UV treatment.

FIG. 11 shows test results on capability of cell attraction on old titanium disks stored in air at different temperatures.

FIG. 12 shows test results on capability of cell attraction on old titanium disks stored in liquid at different temperatures.

### DETAILED DESCRIPTION

In one aspect of the present invention, it is provided a method of placing an implant in a subject, which method comprising:

treating a medical implant with ultraviolet light (UV) in a closed environment,

causing the temperature of the medical implant to be between room temperature (Rt) and about 37° C., and immediately thereafter

placing the implant of a temperature from about the room temperature to about 37° C. in a site in need thereof in the subject.

In some embodiments of the method, the medical implant has a temperature or is exposed to a temperature below room temperature (Rt) or above body temperature prior to receiving the UV treatment.

In some embodiments of the method, the medical implant has a temperature or is exposed to a temperature between 0° C. and about 20° C. prior to receiving the UV treatment.

In some embodiments of the method, the medical implant has a temperature or is exposed to a temperature of 40° C. or above prior to receiving the UV treatment.

In some embodiments of the method, causing the temperature of the medical implant to be between room temperature (Rt) and about 37° C. comprises the act of heating (e.g., heating by the UV treatment) or cooling.

In some embodiments of the method of invention, optionally in combination with any or all of the various above embodiments, the closed environment is a closed chamber.

In some embodiments of the method of invention, optionally in combination with any or all of the various above embodiments, the closed environment is a closed chamber filled with an inert gas, clean air, or carbon-free air.

In some embodiments of the method of invention, optionally in combination with any or all of the various above embodiments, the inert gas comprises N₂, He, or Ar.

In some embodiments of the method of invention, optionally in combination with any or all of the various above embodiments, the medical implant comprises a metallic material.

In some embodiments of the method of invention, optionally in combination with any or all of the various above embodiments, medical implant comprises a surface comprising a micro or nanostructures.

In some embodiments of the method of invention, optionally in combination with any or all of the various above embodiments, the metallic material comprises gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, or combinations thereof.

In some embodiments of the method of invention, optionally in combination with any or all of the various above embodiments, the medical implant is selected from the group consisting of tooth medical implants, jaw bone medical implant, repairing and stabilizing screws, pins, frames (e.g., mesh frames), and plates for bone, spinal medical implants, femoral medical implants, neck medical implants, knee medical implants, wrist medical implants, joint medical implants such as an artificial hip joint, maxillofacial medical implants such as ear and nose medical implants, limb prostheses for conditions resulting from injury and disease, and combinations thereof.

In some embodiments of the method of invention, optionally in combination with any or all of the various above embodiments, the medical implant comprises a non-metallic material.

In some embodiments of the method of invention, optionally in combination with any or all of the various above embodiments, the non-metallic material comprises a polymeric material or a bone cement material.

In some embodiments of the method of invention, optionally in combination with any or all of the various above embodiments, the bone cement material comprises a material selected from the group consisting of polyacrylates, polyesters, bioglass, ceramics, calcium-based materials, calcium phosphate-based materials, and combinations thereof.

In some embodiments of the method of invention, optionally in combination with any or all of the various above embodiments, the bone cement material comprises poly(methyl methacrylate) (PMMA) or methyl methacrylate (MMA).

In some embodiments of the method of invention, optionally in combination with any or all of the various above embodiments, the subject is a mammal.

In some embodiments of the method of invention, optionally in combination with any or all of the various above embodiments, the subject is a human being.

In some embodiments of the method of invention, optionally in combination with any or all of the various above embodiments, the subject has a bone related condition, wherein the method treats or ameliorates the disorder.

In some embodiments of the method of invention, optionally in combination with any or all of the various above embodiments, the bone related condition is a bone related disease or injury.

As used herein, the term treating with an ultraviolet light “UV” can be used interchangeably with the term “light activation,” “light radiation,” “light irradiation,” “UV light activation,” “UV light radiation,” or “UV light irradiation.”

As used herein, the term “UV” or “UV light” shall not encompass a UV laser or UV laser beam. Such UV light does not encompass any UV beam obtained through optical amplification such as those fall within the definition of laser as described in Gould, R. Gordon (1959). “The LASER, Light Amplification by Stimulated Emission of Radiation”. In Franken, P. A. and Sands, R. H. (Eds.). The Ann Arbor Conference on Optical Pumping, the University of Michigan, 15 June through 18 June 1959. p. 128.

As used herein, the term room temperature or Rt generally refers to a temperature of about 25° C. In some embodiments, the term Rt refers to a temperature of 25±1° C.

As used herein, the term body temperature generally refers to a temperature of about 37° C. In some embodiments, the term Rt refers to a temperature from 36° C. to 37.5° C.

As used herein, the term “significantly below room temperature” refers to a temperature of about 20° C. or below, e.g., 0° C., 5° C., 10° C., or 15° C.

As used herein, the term “significantly above room temperature” refers to a temperature of above body temperature, e.g., 38° C., 40° C., 45° C., 50° C., or 55° C.

As used herein, the term “carbon-free air” refers to an air environment that is free from any carbon content or substantially free from any carbon content. Substantially free from any carbon content shall mean an air environment that is removed of at least 90% carbon content (as compared to a normal air environment), which can also be referred to as carbon-minimum air. As used herein, the term “carbon content” refers to any contamination in air containing carbon that is not carbon dioxide. Such contamination can be any organic species, carbon particles, or an inorganic compound in the air that contains carbon.

As used herein, the term “storage in liquid” generally refers to a liquid storage medium for commonly used for storage of medical implants, for example, water or ddH₂O.

### Osteophilic Surface

The term “osteophilic surface” refers to a surface that imparts enhanced tissue integration capabilities to a medical implant. An osteophilic surface can include hydroxyl groups, oxides or both and can have micro or nanostructures. In some embodiments, the nanostructures can include nanoconstructs such as nanospheres, nanocones, nanopyramids, other nanoconstructs or combinations thereof. In some embodiments, the micro or nanoconstructs have a size in the range between about 1 nm and about 1000 um, about 1 nm and about 400 um, about 1 nm and about 100 urn, about 1 nm and about 40 um, about 1 nm and about 10 um, about 1 nm and about 1000 nm, about 1 nm and about 400 nm, between about 1 nm and about 200 nm, between about 1 nm and about 100 nm, between about 10 nm and about 100 nm, between about 10 nm and about 70 nm, between about 20 nm and about 40 nm or between about 20 nm and about 40 nm.

As used herein, the term “tissue integration capability” refers to the ability of a medical implant to be integrated into the tissue of a biological body. The tissue integration capability of a medical implant can be generally measured by several factors, one of which is wettability of the medical implant surface, which reflects the hydrophilicity/oleophilicity (hydrophobicity), or hemophilicity of a medical implant surface. Hydrophilicity and oleophilicity are relative terms and can be measured by, e.g., water contact angle (Oshida Y, et al., J Mater Science 3:306-312 (1992)), and area of water spread (Gifu-kosen on line text, http://www.gifu-nct.ac.jp/elec/tokoro/fft/contact-angle.html). For purposes of the present invention, the hydrophilicity/oleophilicity can be measured by contact angle or area of water spread of a medical implant surface described herein relative to the ones of the control medical implant surfaces. Relative to the medical implant surfaces not treated with the process described herein, a medical implant treated with the process described herein has a substantially lower contact angle or a substantially higher area of water spread.

### Medical Implants

The medical implants described herein with enhanced tissue integration capabilities include any medical implants currently available in medicine or to be introduced in the future. The medical implants can be metallic or non-metallic medical implants. Non-metallic medical implants include, for example, ceramic medical implants, calcium phosphate or polymeric medical implants. Useful polymeric medical implants can be any biocompatible medical implants, e.g., bio-degradable polymeric medical implants. Representative ceramic medical implants include, e.g., bioglass and silicon dioxide medical implants. Calcium phosphate medical implants includes, e.g., hydroxyapatite, tricalcium phosphate (TCP). Exemplary polymeric medical implants include, e.g., poly-lactic-co-glycolic acid (PLGA), polyacrylate such as polymethacrylates and polyacrylates, and poly-lactic acid (PLA) medical implants. In some embodiments, the medical implant described herein can specifically exclude any of the aforementioned materials.

In some embodiments, the medical implant comprises a metallic medical implant and a bone-cement material. The bone cement material can be any bone cement material known in the art. Some representative bone cement materials include, but are not limited to, polyacrylate or polymethacrylate based materials such as poly(methyl methacrylate) (PMMA)/methyl methacrylate (MMA), polyester based materials such as PLA or PLGA, bioglass, ceramics, calcium phosphate-based materials, calcium-based materials, and combinations thereof. In some embodiments, the medical implant can include any polymer described below. In some embodiments, the medical implant described herein can specifically exclude any of the aforementioned materials.

The metallic medical implants described herein include titanium medical implants and non-titanium medical implants. Titanium medical implants include tooth or bone replacements made of titanium or an alloy that includes titanium. Titanium bone replacements include, e.g., knee joint and hip joint prostheses, femoral neck replacement, spine replacement and repair, neck bone replacement and repair, jaw bone repair, fixation and augmentation, transplanted bone fixation, and other limb prostheses. None-titanium metallic medical implants include tooth or bone medical implants made of gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, e.g., stainless steel, or combinations thereof. Some examples of alloys are titanium-nickel allows such as nitanol, chromium-cobalt alloys, stainless steel, or combinations thereof. In some embodiments, the metallic medical implant can specifically exclude any of the aforementioned metals.

The medical implant described herein can be porous or non-porous medical implants. Porous medical implants can impart better tissue integration while non-porous medical implants can impart better mechanical strength.

The medical implants can be metallic medical implants or non-metallic medical implants. In some embodiments, the medical implants are metallic medical implants such as titanium medical implants, e.g., titanium medical implants for replacing missing teeth (dental medical implants) or fixing diseased, fractured or transplanted bone. Other exemplary metallic medical implants include, but are not limited to, titanium alloy medical implants, chromium-cobalt alloy medical implants, platinum and platinum alloy medical implants, nickel and nickel alloy medical implants, stainless steel medical implants, zirconium, chromium-cobalt alloy, gold or gold alloy medical implants, and aluminum or aluminum alloy medical implants.

The medical implants provided herein can be subjected to various established surface treatments to increase surface area or surface roughness for better tissue integration or tissue attachment. Representative surface treatments include, but are not limited to, physical treatments and chemical treatments. Physical treatments include, e.g., machined process, sandblasting process, metallic deposition, non-metallic deposition (e.g., apatite deposition), or combinations thereof. Chemical treatment includes, e.g., etching using a chemical agent such as an acid, base (e.g., alkaline treatment), oxidation (e.g., heating oxidation and anodic oxidation), and combinations thereof. For example, a metallic medical implant can form different surface topographies by a machined process or an acid-etching process.

### Polymers

The polymers can be any polymer commonly used in the medical device industry.

The polymers can be biocompatible or non-biocompatible. In some embodiments, the polymer can be poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide-co-lactic acid) (PEO/PLA)), polyalkylene oxides such as poly(ethylene oxide), poly(propylene oxide), poly(ether ester), polyalkylene oxalates, phosphoryl choline containing polymer, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, methacrylate polymers containing 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), molecules such as collagen, chitosan, alginate, fibrin, fibrinogen, cellulose, starch, dextran, dextrin, hyaluronic acid, fragments and derivatives of hyaluronic acid, heparin, fragments and derivatives of heparin, glycosamino glycan (GAG), GAG derivatives, polysaccharide, elastin, elastin protein mimetics, or combinations thereof. Some examples of elastin protein mimetics include (LGGVG)ₙ, (VPGVG)ₙ, Val-Pro-Gly-Val-Gly, or synthetic biomimetic poly(L-glytanmate)-b-poly(2-acryloyloxyethyllactoside)-b-poly(l-glutamate) triblock copolymer.

In some embodiments, the polymer can be poly(ethylene-co-vinyl alcohol), poly(methoxyethyl methacrylate), poly(dihydroxylpropyl methacrylate), polymethacrylamide, aliphatic polyurethane, aromatic polyurethane, nitrocellulose, poly(ester amide benzyl), co-poly-{[N,N′-sebacoyl-bis-(L-leucine)-1,6-hexylene diester]o.75-[N,N′-sebacoyl-L-lysine benzyl cstcr]o,25} (PEA-Bz), co-poly-{[N,N′-sebacoyl-bis-(L-leucine)-1,6-hexylene diester]o.75-[N,N′-sebacoyl-L-lysine-4-amino-TEMPO amide]0.25} (PEA-TEMPO), aliphatic polyester, aromatic polyester, fluorinated polymers such as poly(vinylidene fluoride-co-hexafluoropropylene), poly(vinylidene fluoride) (PVDF), and Teflon™ (polytetrafluoroethylene), a biopolymer such as elastin mimetic protein polymer, star or hyper-branched SIBS (styrene-block-isobutylene-block-styrene), or combinations thereof. In some embodiments, where the polymer is a copolymer, it can be a block copolymer that can be, e.g., di-, tri-, tetra-, or oligo-block copolymers or a random copolymer. In some embodiments, the polymer can also be branched polymers such as star polymers.

In some embodiments, a UV-transmitting material having the features described herein can exclude any one of the aforementioned polymers.

As used herein, the terms poly(D,L-lactide), poly(L-lactide), poly(D,L-lactide-co-glycolide), and poly(L-lactide-co-glycolide) can be used interchangeably with the terms poly(D,L-lactic acid), poly(L-lactic acid), poly(D,L-lactic acid-co-glycolic acid), or poly(L-lactic acid-co-glycolic acid), respectively.

### Medical Use

The medical implants provided herein can be used for treating, preventing, ameliorating, correcting, or reducing the symptoms of a medical condition by medical implanting the medical implants in a mammalian subject. The mammalian subject can be a human being or a veterinary animal such as a dog, a cat, a horse, a cow, a bull, or a monkey.

Representative medical conditions that can be treated or prevented using the medical implants provided herein include, but are not limited to, missing teeth or bone related medical conditions such as femoral neck fracture, missing teeth, a need for orthodontic anchorage or bone related medical conditions such as femoral neck fracture, neck bone fracture, wrist fracture, spine fracture/disorder or spinal disk displacement, fracture or degenerative changes of joints such as knee joint arthritis, bone and other tissue defect or recession caused by a disorder or body condition such as, e.g., cancer, injury, systemic metabolism, infection or aging, and combinations thereof.

In some embodiments, the medical implants provided herein can be used to treat, prevent, ameliorate, or reduce symptoms of a medical condition such as missing teeth, a need for orthodontic anchorage or bone related medical conditions such as femoral neck fracture, neck bone fracture, wrist fracture, spine fracture/disorder or spinal disk displacement, fracture or degenerative changes of joints such as knee joint arthritis, bone and other tissue defect or recession caused by a body condition or disorder such as cancer, injury, systemic metabolism, infection and aging, limb amputation resulting from injuries and diseases, and combinations thereof.

### EXAMPLES

The following examples illustrate, and shall not be construed to limit, the embodiments of the present invention.

### Example 1

### Reactivation of High Energy and Cell-attractive Implant Materials

### Summary

Here, we have demonstrated that temperature change deviated from the room temperature degrades the superhydrophilicity and high bioactivity of titanium implants immediately, regardless of whether they are new surfaces or UV-treated surfaces. Given the above mentioned fact of the current distribution and sales system of implant products, this uncovered a new fact that the delivery of new titanium surfaces and UV-treated titanium surfaces, while maintaining their high energy and bioactivity, is virtually impossible and that treating implants with UV on site at the peripheral users' level immediately before the use to the patients is the only effective measure to ensure the high energy and bioactive surfaces. We then have demonstrated that UV treatment is capable to recover the superhydrophilicity and high bioactivity that had been rapidly impaired or lost by temperature changes.

UV light treatment has been used for medical purpose because of its bacteriocidal ability. The effect of UV treatment in increasing the bioactivity of implant materials by removing surface impurities, such as hydrocarbons, was reported. However, the finding on the effectiveness of UV treatment to re-activate the high energy and bioactivity implant surfaces than are abrogated by temperature change is novel, which for the first time has made us realize that it ruins the advantages of UV treatment when the UV treatment is carried out at the manufactures level and that at the same time opened a novel avenue of effective UV application at the users level immediately before the use for the patients. The demonstrated effectiveness and thereby suggested technological and procedural matters on the use of UV treatment will provide a definitive solution for the current problems and significant advantage in its clinical and commercial application to enhance the currently used implant devices in dental and orthopedic fields.

### Results

Temperature Change During Air Storage Immediately Reduces Hydrophilicity of UV-induced High Energy Titanium

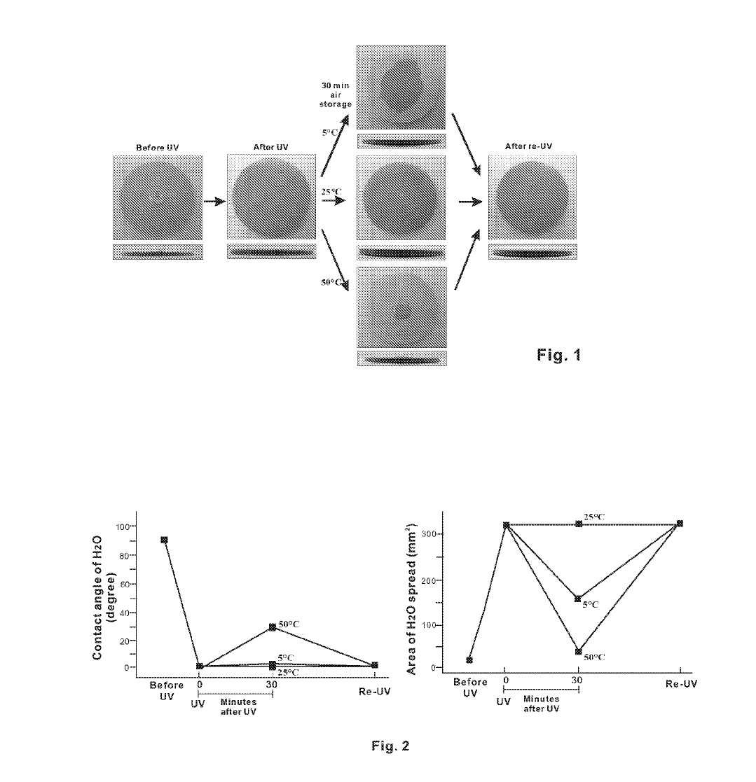

First, sufficiently old titanium disks with hydrophobic nature whose contact angle of 10 μl ddH₂O was >60° was treated with UV light. The UV-treated titanium showed the superhydrophilicity where the contact angle of ddH₂O was 0° and the area of 10 μl ddH₂O spread was 308±6 mm² (FIGS. 1 and 2). The UV-treated titanium disks were stored for 30 min in either 0°, 25° (considered as room temperature), or 50° air in a sealed condition. While the titanium disks stored in 25° air remained superhydrophilic with the equivalent contact angle and spread area of 10 μl ddH₂O as those immediately after UV treatment, the titanium disks stored in 5° and 50° air showed a significant reduction in their hydrophilicity. The titanium disks stored in in 5° air showed a 10 μl ddH₂O spread of 152±25 mm². The titanium disks stored in in 50° air showed a 10 μl ddH₂O spread of 41±5 mm² and its contact angle of 31±3.5°.

Reduced Hydrophilicity by Temperature Change was Fully Recovered by Re-UV Treatment

The above mentioned titanium surfaces with temperature change-reduced hydrophilicity was re-treated with UV light. All of the re-UV-treated titanium surfaces showed a fully-regenerated superhydrophilicity with its contact angle of 0° and ddH₂O spread of 307±6 mm² (FIGS. 1 and 2).

Temperature Change During Liquid Storage Reduces Hydrophilicity of UV-induced High Energy Titanium

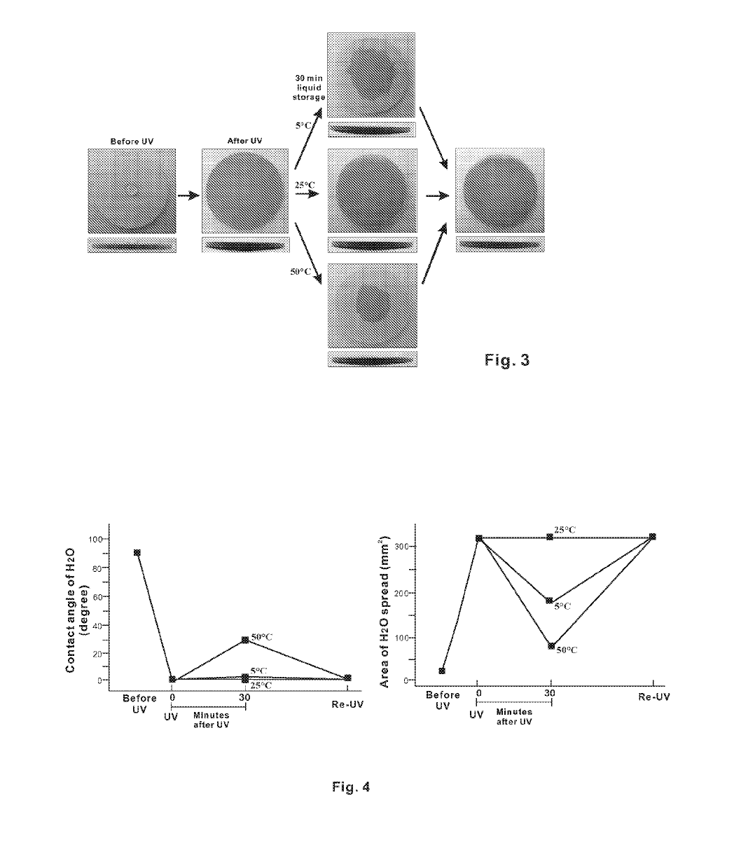

Next we examine the effect of temperature change of titanium when it is stored in liquid. Sufficiently old titanium disks with hydrophobic nature whose contact angle of 10 μl ddH₂O was >60° was treated with UV light. The UV-treated titanium disks showed the superhydrophilicity where the contact angle of ddH₂O was 0° and the area of 10 μl ddH₂O spread was 308±4 mm (FIGS. 3 and 4). The UV-treated titanium disks were stored for 30 min in either 0°, 25° (considered as room temperature), or 50° ddH₂O. While the titanium disks stored in 25° water remained superhydrophilic with the equivalent contact angle and spread area of 10 μl ddH₂O as those immediately after UV treatment, the titanium disks stored in 5° and 50° water showed a significant reduction in their hydrophilicity. The titanium disks stored in in 5° air showed a 10 μl ddH₂O spread of 180±16 mm². The titanium disks stored in in 50° air showed a ddH₂O spread of 75±9 mm².

Reduced Hydrophilicity by Liquid Temperature Change was Fully Recovered by Re-UV Treatment

The above mentioned titanium surfaces with temperature change-reduced hydrophilicity was re-treated with UV light. All of the re-UV-treated titanium surfaces 10 showed a fully-regenerated superhydrophilicity with its contact angle of 0° and ddH2O spread of 309±5 mm² (FIGS. 3 and 4).

Temperature Change During Air Storage Reduces Hydrophilicity of Newly Prepared High Energy Titanium

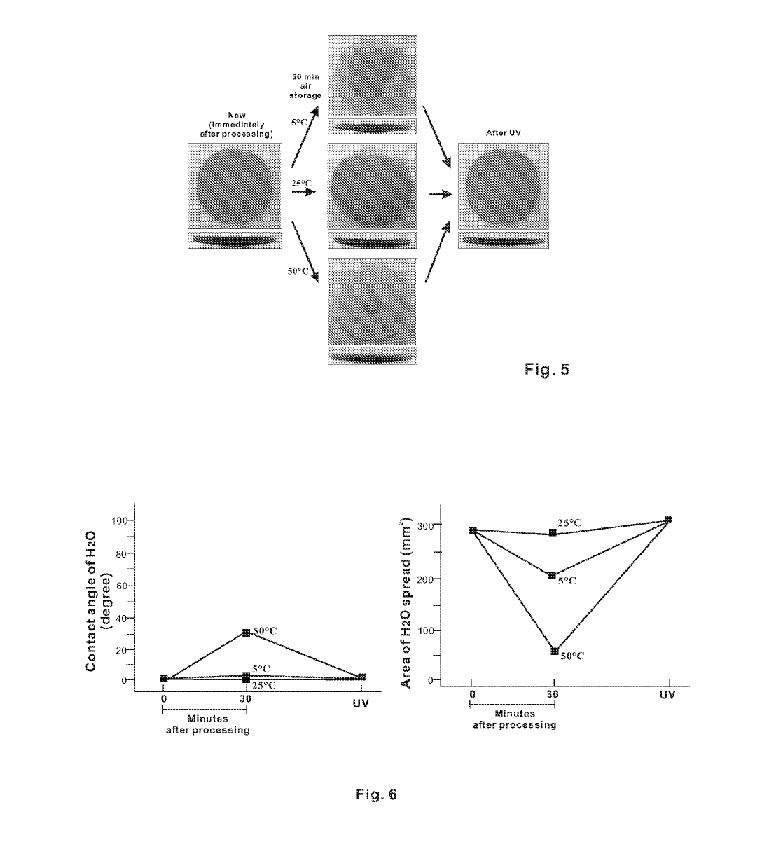

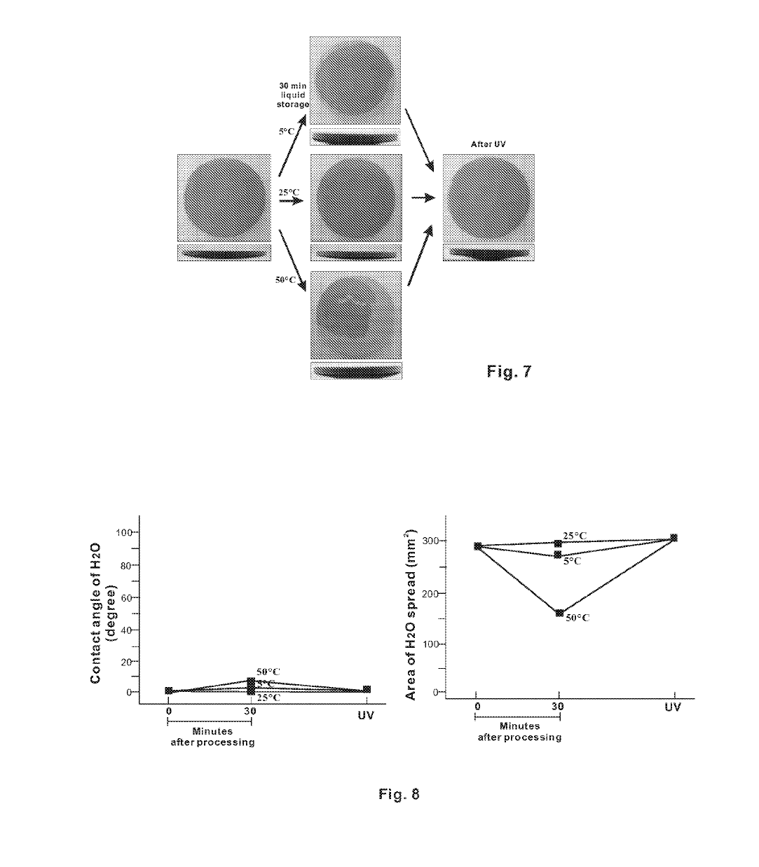

We next performed similar experiments using fresh titanium surfaces, which are 15 new titanium surfaces immediately after processing. The acid-etched titanium disks were made and their hydrophilicity was evaluated immediately. All of these new titanium surfaces showed the superhydrophilicity where the contact angle of ddH₂O was 0° and the area of 10 μl ddH₂O spread was 295±5 mm (FIGS. 5 and 6). The new titanium disks were stored for 30 min in either 0°, 25° (considered as room temperature), or 50° air. While the titanium disks stored in 25° air remained superhydrophilic with the equivalent contact angle and spread area of 10 μl ddH₂O as those immediately after processing, the new titanium disks stored in 5° and 50° air showed a significant reduction in their hydrophilicity. The titanium disks stored in in 5° air showed a 10 μl ddH₂O spread of 225±18 mm². The titanium disks stored in in 50° air showed a 10 μl ddH₂O spread of 53±8 mm² and its contact angle of 35±70°.

Reduced Hydrophilicity of New Titanium Surfaces by Temperature Change During Air Storage was Fully Recovered by UV Treatment

The titanium surfaces having their hydrophilicity reduced during air storage in high and low temperature was re-treated with UV light. All of the re-UV-treated titanium surfaces fully recovered superhydrophilicity with its contact angle of 0° and ddH₂O spread of 308±5 mm² (FIGS. 5 and 6).

Temperature Change During Liquid Storage Reduces Hydrophilicity of Newly Prepared High Energy Titanium

We next stored new titanium surfaces in liquid at various temperature: 0°, 25° (considered as room temperature), or 50° ddH₂O. While the titanium disks stored in 25° water remained superhydrophilic with the equivalent contact angle and spread area of 10 μl ddH₂O as those immediately after processing, the new titanium surfaces stored in 5° and 50° water showed a significant reduction in their hydrophilicity. The titanium disks stored in in 5° air showed a 10 μl ddH₂O spread of 275±22 mm². The titanium disks stored in 50° air showed the contact angle of 5±2° and the area of 10 μl ddH₂O spread of 168±19 mm².

Reduced Hydrophilicity by Temperature Change During Liquid Storage was Fully Recovered 10 by UV Treatment

The new titanium surfaces having their hydrophilicity reduced during liquid storage in high and low temperature was treated with UV light. All of the UV-treated titanium surfaces fully recovered superhydrophilicity with its contact angle of 0° and ddH₂O spread of 310±2 mm².

Temperature Change During Air Storage Immediately Reduces Cell Attraction Capability of UV-induced Bioactive Titanium

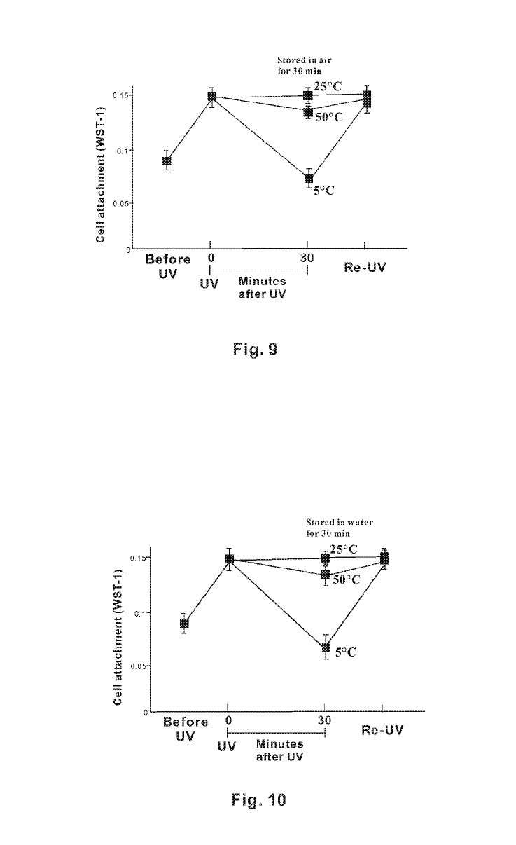

First, old titanium disks with and without UV treatment were compared for their capability of cell attraction. After 2 h of incubation, adhered cells were quantified using WST-1 assay (FIG. 9). UV treatment of old titanium disks significantly increased the 20 number of attached cells during a 2-h incubation. Next, the UV-treated titanium disks were stored for 30 min in air at different temperature of 5°, 25°, or 50°. The number of attached cells was significantly reduced on titanium disks stored at 5° C. and 50° C. (p<0.05), while it did not change on titanium disks stored at 25° C.

Re-UV Treatment Recovers the Temperature Change-induced Reduction of Cell Attraction Capability of UV-treated Titanium

The UV-treated titanium disks stored in different conditions were re-treated with UV and their cell attraction capability was evaluated (FIG. 9). The reduced number of attached cells on titanium disk stored at 5° C. and 50° C. was fully recovered by the re-UV treatment to the equivalent level of the titanium disks stored at 25° C. and immediately after the first UV treatment.

Re-UV Treatment was Effective in Recovering the Reduced Cell Attraction Capability of UV-treated Titanium after Storing in High- and Low-temperature Liquid

Likewise, storage in liquid condition that was higher and lower temperature than 25° C. significantly reduced the number of attached cells (p<0.05; FIG. 10). The reduced cell attachment capability was, however, was fully brought back to the level of the 25° C. storage and the state before such storage.

Temperature Change During Air Storage Immediately Reduced Cell Attraction Capability of New Titanium

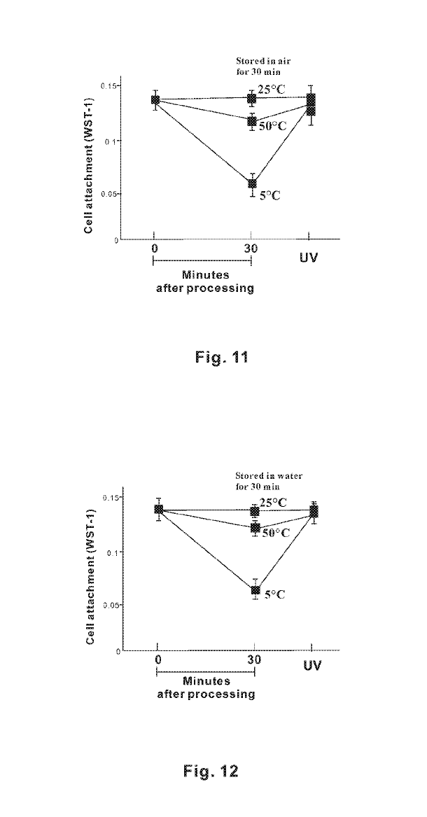

Titanium disks were newly prepared and stored for 30 min in air at different temperature of 5°, 25°, or 50°. Two hours after seeding cells onto these titanium surfaces, adhered cells were quantified using WST-1 assay (FIG. 11). The number of attached cells was significantly reduced on titanium disks stored at 5° C. and 50° C. air (p<0.05), while it did not change on titanium disks stored at 25° C. air.

UV Treatment Recovers the Temperature-induced Reduction of Cell Attraction Capability of New Titanium

The new titanium disks stored in different conditions were treated with UV and their cell attraction capability was evaluated (FIG. 11). The reduced number of attached cells on titanium disk stored at 5° C. and 50° C. was fully recovered by UV treatment to the equivalent level of the titanium disks stored at 25° C. and the level before the storage.

UV Treatment was Effective in Recovering the Reduced Cell Attraction Capability of New Titanium After Storing in High- and Low-temperature Liquid

Likewise, storage in ddH₂O that was higher and lower temperature than 25° C. significantly reduced the number of attached cells to new titanium surfaces (p<0.05; FIG. 12). The reduced cell attachment capability was, however, was fully brought back by UV treatment to the level of 25° C. ddH₂O storage and the state before such storage.

### Materials and Methods

Titanium Sample

Disks (20 mm in diameter and 1.0 mm in thickness) made of commercially pure titanium (Grade 2) were used. Titanium disks were acid-etched with 67% H₂SO₄ at 120° C. for 75 seconds to simulate the most commonly used surface in the implant market. UV treatment was performed for 20 min using UV light; intensity, ca. 0.5 mW/cm² (λ=360±20 nm) and 1.5 mW/cm² (λ=250±20 nm). The temperature of the titanium disks was measured by surface thermometer (AD-5601A, AND Inc., Tokyo, Japan).

Bone-forming Cell (Osteoblast) Cell Culture

Bone marrow cells isolated from the femur of 8-week-old male Sprague-Dawley rats were placed into alpha-modified Eagle's medium supplemented with 15% fetal bovine serum, 50 mg/ml ascorbic acid, 10⁻⁸M dexamethasone, 10 mM Na-β-glycerophosphate and Antibiotic-antimycotic solution containing 10000 units/ml Penicillin G sodium, 10000 mg/ml Streptomycin sulfate and 25 mg/ml Amphotericin B. Cells were incubated in a humidified atmosphere of 95% air, 5% C02 at 37° C. At 80% confluency, the cells were detached using 0.25% Trypsin-1 mM EDTA-4Na and seeded onto titanium disks at a density of 3×10⁴ cells/cm².

Cell Attachment

Initial attachment of cells was evaluated by measuring the quantity of the cells attached to titanium substrates after 2 hours of incubation. The quantification was performed using WST-1 based colorimetry (WST-1, Roche Applied Science, Mannnheim, Germany). The culture well was incubated at 37° C. for 4 hours with 100 μl tetrazolium salt (WST-1) reagent. The amount of formazan product was measured using an ELISA reader at 420 nm.

Statistical Analysis

ANOVA was used to examine differences in variables between differently treated titanium disks. If necessary, a post-hoc Bonferroni test was used as a multiple comparisons test; p<0.05 was considered significant.

### REFERENCES

[1] Ray N F, Chan J K, Thamer M, Melton L J, 3rd. Medical expenditures for the treatment of osteoporotic fractures in the United States in 1995: report from the National Osteoporosis Foundation. J Bone Miner Res 1997;12:24.
[2] Hudson J I, Kenzora J E, Hebel J R, Gardner J F, Scherlis L, Epstein R S, Magaziner J S. Eight-year outcome associated with clinical options in the management of femoral neck fractures. Clin Orthop Relat Res 1998:59.
[3] Lu-Yao G L, Keller R B, Littenberg B, Wennberg J E. Outcomes after displaced fractures of the femoral neck. A meta-analysis of one hundred and six published reports. J Bone Joint Surg Am 1994;76:15.
[4] Tidermark J, Ponzer S, Svensson O, Soderqvist A, Tornkvist H. Internal fixation compared with total hip replacement for displaced femoral neck fractures in the elderly. A randomised, controlled trial. J Bone Joint Surg Br 2003;85:380.
[5] Ravikumar K J, Marsh G. Internal fixation versus hemiarthroplasty versus total hip arthroplasty for displaced subcapital fractures of femur—13 year results of a prospective randomised study. Injury 2000;31:793.
[6] van Steenberghe D, Jacobs R, Desnyder M, Maffei G, Quirynen M. The relative impact of local and endogenous patient-related factors on implant failure up to the abutment stage. Clin Oral Implants Res 2002; 13:617.
[7] Ozawa S, Ogawa T, Iida K, Sukotjo C, Hasegawa H, Nishimura RD, Nishimura I. Ovariectomy hinders the early stage of bone-implant integration: histomorphometric, biomechanical, and molecular analyses. Bone 2002;30:137.
[8] Nevins M L, Karimbux N Y, Weber H P, Giannobile W V, Fiorellini J P. Wound healing around endosseous implants in experimental diabetes. Int J Oral Maxillofac Implants 1998; 13:620.
[9] Zhang H, Lewis C G, Aronow M S, Gronowicz G A. The effects of patient age on human osteoblasts' response to Ti-6A1-4V implants in vitro. J Orthop Res 2004;22:30.
[10] Takeshita F, Murai K, Ayukawa Y, Suetsugu T. Effects of aging on titanium implants inserted into the tibiae of female rats using light microscopy, SEM, and image processing. J Biomed Mater Res 1997;34:1.
[11] Yamazaki M, Shirota T, Tokugawa Y, Motohashi M, Ohno K, Michi K, Yamaguchi A. Bone reactions to titanium screw implants in ovariectomized animals. Oral Surg Oral Med Oral Pathol Oral Radiol Endod 1999;87:411.
[12] Att W, Hori N, Takeuchi M, Ouyang J, Yang Y, Anpo M, Ogawa T. Time-dependent degradation of titanium osteoconductivity: an implication of biological aging of implant materials. Biomaterials 2009;30:5352.
[13] Att W, Hori N, Iwasa F, Yamada M, Ueno T, Ogawa T. The effect of UV-photofunctionalization on the time-related bioactivity of titanium and chromium-cobalt alloys. Biomaterials 2009;30:4268.
[14] Aita H, Hori N, Takeuchi M, Suzuki T, Yamada M, Anpo M, Ogawa T. The effect of ultraviolet functionalization of titanium on integration with bone. Biomaterials 2009;30:1015.
[15] Suzuki T, Hori N, Att W, Kubo K, Iwasa F, Ueno T, Maeda H, Ogawa T. Ultraviolet treatment overcomes time-related degrading bioactivity of titanium. Tissue Eng Part A 2009; 15:3679.
   While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A method of placing a medical implant in a subject, comprising:
treating a medical implant with ultraviolet light (UV) in a closed environment,
causing the temperature of the medical implant to be between room temperature (Rt) and about 37° C., and immediately thereafter
placing the implant of a temperature from about the room temperature to about 37° C. in a site in need thereof in the subject.

2. The method of claim 1, wherein the medical implant has a temperature or is exposed to a temperature below room temperature (Rt) or above body temperature, prior to the UV treatment.

3. The method according to claim 1, wherein the medical implant has a temperature between 0° C. and about 20° C., prior to receiving the UV treatment.

4. The method according to claim 1, wherein the medical implant has a temperature of 40° C. or above, prior to receiving the UV treatment.

5. The method of claim 1, wherein the closed environment is a closed chamber.

6. The method of claim 1, wherein the closed environment is a closed chamber filled with an inert gas, clean air, or carbon-free air.

7. The method of claim 5, wherein the inert gas comprises N₂, He, or Ar.

8. The method of claim 1, wherein the medical implant comprises a metallic material.

9. The method of claim 1, wherein medical implant comprises a surface comprising a microstructure or a nanostructure.

10. The method of claim 8, wherein the metallic material comprises gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, or combinations thereof.

11. The method of claim 10, wherein the medical implant is selected from the group consisting of tooth medical implants, jaw bone medical implant, repairing and stabilizing screws, pins, frames, and plates for bone, spinal medical implants, femoral medical implants, neck medical implants, knee medical implants, wrist medical implants, joint medical implants, an artificial hip joint, maxillofacial medical implants, ear implants, nose medical implants, limb prostheses for conditions resulting from injury and disease, and combinations thereof.

12. The method of claim 1, wherein the medical implant comprises a non-metallic material.

13. The method of claim 12, wherein the non-metallic material comprises a polymeric material or a bone cement material.

14. The method of claim 13, wherein the bone cement material comprises a material selected from the group consisting of polyacrylates, polyesters, bioglass, ceramics, calcium-based materials, calcium phosphate-based materials, and combinations thereof.

15. The method of claim 14, wherein the bone cement material comprises poly(methyl methacrylate) (PMMA) or methyl methacrylate (MMA).

16. The method of claim 1, wherein the subject is a mammal.

17. The method of claim 1, wherein the subject is a human being.

18. The method of claim 1, wherein the subject has a bone related condition, wherein the method treats or ameliorates the disorder.

19. The method of claim of claim 18, wherein the bone related condition is a bone related disease or injury.
